(19) United States
(12) Patent Application Publication
(10) Pub. No.: US 2007/0213774 A1
(43) Pub. Date: Sep. 13, 2007

(54) DEFIBRILLATION THRESHOLD TESTING SYSTEM WITH AUTOMATED CONTROL OF EXTERNAL DEFIBRILLATOR

(76) Inventor: Nader Kameli, Hugo, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/276,751

(22) Filed: Mar. 13, 2006

(51) Int. Cl. A61N 1/39

(52) U.S. Cl. 607/5; 607/8

(57) ABSTRACT

A system for conducting a defibrillation threshold (DFT) test for programming an implantable medical device such as an implantable cardioverter defibrillator (ICD) during its implantation includes an external defibrillator as a back up device. The system reduces the risk to the patient by performing an automated external defibrillation procedure when external defibrillation is needed to recover the patient from an arrhythmia episode induced for the DFT test.

### TECHNICAL FIELD

This document generally relates to cardiac rhythm management (CRM) systems and particularly to a system for testing defibrillation threshold (DFT) during implantation of an implantable medical device such as an implantable cardioverter defibrillator (ICD).

### BACKGROUND

Tachyarrhythmias are abnormal heart rhythms characterized by a rapid heart rate. Tachyarrhythmias generally include supraventricular tachyarrhythmia (SVT, including atrial tachyarrhythmia, AT) and ventricular tachyarrhythmia (VT). Fibrillation is a form of tachyarrhythmia further characterized by an irregular heart rhythm. In a normal heart, the sinoatrial node, the heart's predominant natural pacemaker, generates electrical impulses, called action potentials, that propagate through an electrical conduction system to the atria and then to the ventricles of the heart to excite the myocardial tissues. The atria and ventricles contract in the normal atrio-ventricular sequence and synchrony to result in efficient blood-pumping functions indicated by a normal hemodynamic performance. VT occurs when the electrical impulses propagate along a pathologically formed self-sustaining conductive loop within the ventricles or when a natural pacemaker in a ventricle usurps control of the heart rate from the sinoatrial node. When the atria and the ventricles become dissociated during VT, the ventricles may contract before they are properly filed with blood, resulting in diminished blood flow throughout the body. This condition becomes life-threatening when the brain is deprived of sufficient oxygen supply. Ventricular fibrillation (VF), in particular, stops blood flow within seconds and, if not timely and effectively treated, causes immediate death. In very few instances a heart recovers from VF without treatment.

Implantable cardioverter defibrillators (ICDs) are used to treat most tachyarrhythmias, including AT, VT, and VF. An ICD is an implantable medical device that delivers a defibrillation pulse (shock) to terminate a detected tachyarrhythmia episode. The electric defibrillation pulse depolarizes portions of the myocardium and renders it refractory. During the implantation of an ICD to a patient anticipating tachyarrhythmia episodes, a defibrillation threshold (DFT) test is performed to determine the DFT, which is the energy level of the defibrillation pulse required to terminate a tachyarrhythmia episode of that patient. The energy level of each defibrillation pulse is then programmed to a level exceeding the DFT by a safety margin. The programmable energy level for each defibrillation pulse is limited to the ICD energy level.

During a DFT test, several energy levels are programmed to be tested. A VF episode is induced, such as by delivering a defibrillation pulse during a ventricular repolarization (on a T wave). A defibrillation pulse at the programmed minimum energy level is delivered from the ICD to terminate the induced VF. If the defibrillation pulse does not terminate the VF, another defibrillation pulse at the programmed next, stepped-up energy level is delivered. The delivery of the defibrillation pulses is repeated until the VF episode is terminated or until the programmed maximum energy level has been tested. If the VF episode is terminated, the last energy level (the energy level of the defibrillation pulse that terminated the VF episode) is the DFT. If the VF episode is not terminated, the VF episode is to be terminated without using the ICD. This may happen, for example, when the patient's DFT exceeds the programmed maximum energy level, or when defibrillation electrodes are not properly positioned in the patient. Thus, for the safety of the patient, there is a need for a device capable of terminating an arrhythmia episode, such as a VF episode, induced for a DFT test, in addition to the ICD.

### SUMMARY

A system for conducting a defibrillation threshold (DFT) test for programming an implantable medical device such as an implantable cardioverter defibrillator (ICD) during its implantation includes an external defibrillator as a back up device. The system reduces the risk to the patient by performing an automated external defibrillation procedure when external defibrillation is needed to recover the patient from an arrhythmia episode induced for the DFT test.

In one embodiment, a system for conducting a DFT test for programming an implantable medical device includes an external defibrillator and a medical device programmer. The external defibrillator is communicatively coupled to the medical device programmer. The external defibrillator delivers one or more external defibrillation pulses in response to an external defibrillation command. The medical device programmer programs the implantable medical device to perform the DFT test and produces the external defibrillation command if an arrhythmia episode induced prior to the DFT test sustains after the DFT test.

In one embodiment, a method for conducting a DFT test for programming an implantable medical device is provided. The DFT test is performed using the implantable medical device. Whether an arrhythmia episode induced prior to the DFT test sustains following the DFT test is determined using a medical device programmer. If the induced arrhythmia episode sustains following the DFT test, an external defibrillation command is produced using the medical device programmer. A delivery of one or more external defibrillation pulses is controlled according to an automated external defibrillation procedure in response to the external defibrillation command using at least one of the medical device programmer and an external defibrillator. The one or more external defibrillation pulses are delivered from the external defibrillator.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of a cardiac rhythm management (CRM) system and portions of the environment in which the CRM system operates.

FIG. 2 is an illustration of another embodiment of the CRM system and portions of the environment in which the CRM system operates.

FIG. 3 is an illustration of another embodiment of the CRM system and portions of the environment in which the CRM system operates.

FIG. 4 is a block diagram illustrating an embodiment of an implantable cardioverter defibrillator (ICD) of the CRM system.

FIGS. 5A-B are each a portion of a block diagram illustrating an embodiment of an external defibrillator system and a programmer system of the CRM system.

FIGS. 6A-B are each a portion of a block diagram illustrating another embodiment of the external defibrillator system and the programmer system.

FIG. 7 is a block diagram illustrating an embodiment of an arrhythmia induction system of the CRM system.

FIG. 8 is a flow chart illustrating an embodiment of a method for conducting a defibrillation threshold (DFT) test using an AED as a backup defibrillator.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses a system for conducting a DFT test for programming an ICD during its implantation. The system includes an external defibrillator as a back up device. The external defibrillator is automatically activated to defibrillate the patient when the ICD fails to recover the patient from an arrhythmia episode induced for the DFT. A medical device programmer of the system controls the ICD to perform the DFT test and controls the external defibrillator to recover the patient from the induced arrhythmia episode if the induced arrhythmia episode sustains after the DFT test or has lasted beyond a predetermined period of time (such as about 30-40 seconds). In one embodiment, the external defibrillator is an automated external defibrillator (AED) that delivers defibrillation pulses and performs an automated external defibrillation procedure to control the delivery of the defibrillation pulses in response to a command issued by the medical device programmer. In another embodiment, the external defibrillator delivers the defibrillation pulses, and the medical device programmer performs the automated external defibrillation procedure to control the delivery of the defibrillation pulses from the external defibrillator. In a further embodiment, the system includes an external arrhythmia induction system for inducting the arrhythmia episode prior to the DFT test.

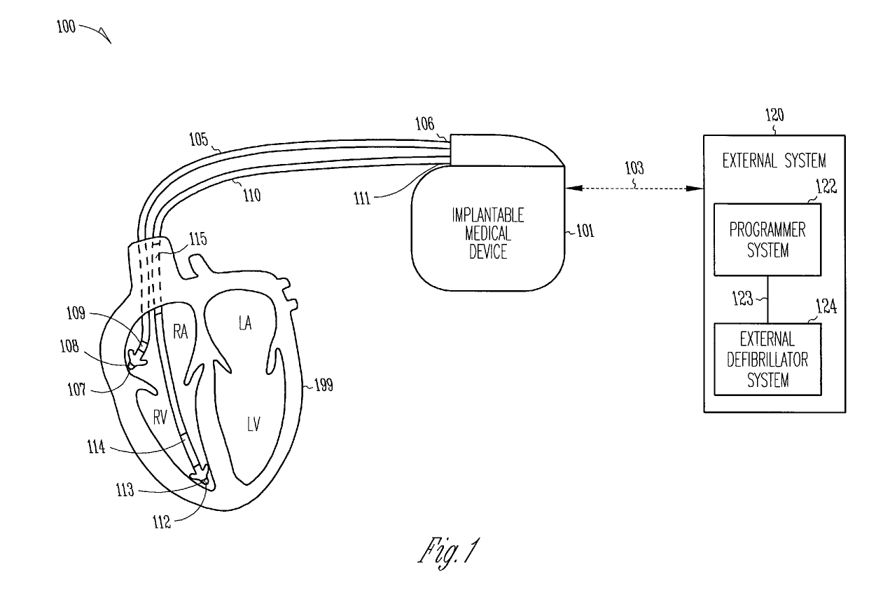

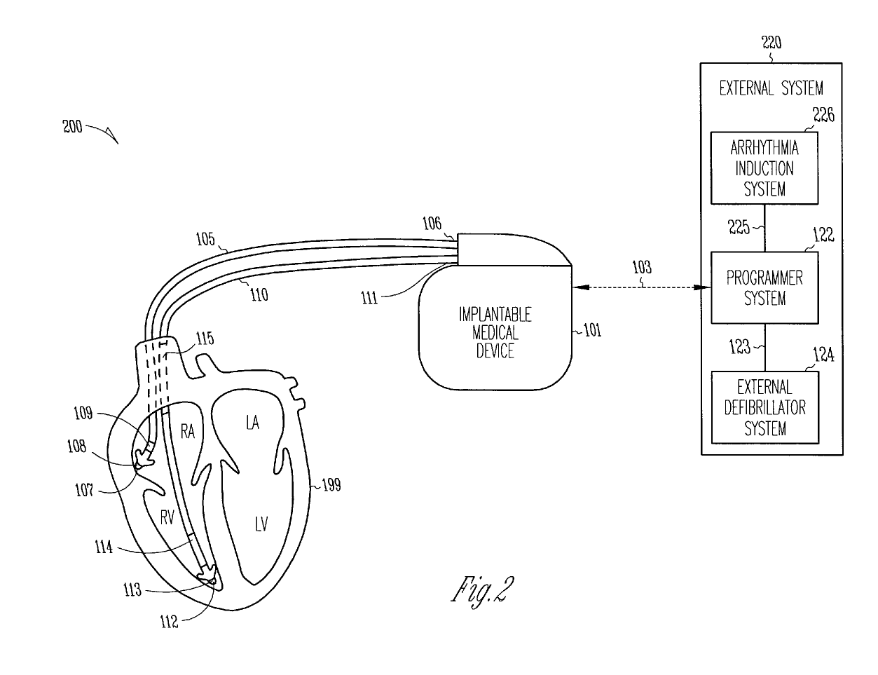

FIG. 1 is an illustration of an embodiment of a CRM system 100 and portions of the environment in which CRM system 100 operates. CRM system 100 includes an implantable medical device 101 that is electrically coupled to a heart 199 through one or more electrodes, such as on leads 105 and 110. An external system 120 communicates with implantable medical device 101 via a telemetry link 103.

Implantable medical device 101 delivers anti-tachyarrhythmia therapies including cardioversion and defibrillation therapies. In one embodiment, implantable medical device 101 is an ICD. In a specific embodiment, the ICD includes a cardiac pacemaker that delivers cardiac pacing pulses. In various embodiments, in addition to a cardioverter defibrillator, implantable medical device 101 includes one or more of other monitoring and/or therapeutic devices such as a cardiac pacemaker, a neural stimulator, a drug delivery device, and a biological therapy device. Implantable medical device 101 includes a hermetically sealed can housing an electronic circuit that senses physiological signals and delivers therapeutic electrical pulses. The hermetically sealed can also functions as an electrode for sensing and/or pulse delivery purposes. In one embodiment, as illustrated in FIG. 1, the electronic circuit senses at least an atrial electrogram and a ventricular electrogram from heart 199 and delivers pacing, cardioversion, and/or defibrillation pulses to heart 199. Lead 105 is a pacing lead that includes a proximal end 106 connected to implantable medical device 101 and a distal end 107 placed in the right atrium (RA) of heart 199. A pacing-sensing electrode 108 (referred to as the “RA tip” electrode) is located at distal end 107. Another pacing-sensing electrode 109 (referred to as the “RA ring” electrode) is located near distal end 107. Electrodes 108 and 109 are electronically connected to implantable medical device 101 via separate conductors in lead 105 to allow sensing of the atrial electrogram and/or delivery of atrial pacing pulses. Lead 110 is a defibrillation lead that includes a proximal end 111 connected to implantable medical device 101 and a distal end 112 placed in the right ventricle (RV) of heart 199. A pacing-sensing electrode 113 (referred to as the “RV tip” electrode) is located at distal end 112. A defibrillation electrode 114 (referred to as the “RV coil” electrode) is located near distal end 112 but electrically separated from pacing-sensing electrode 113. Another defibrillation electrode 115 (referred to as the “SVC coil” electrode) is located at a distance from distal end 112 for placement in the superior vena cava (SVC). In one embodiment, electrode 115 is electrically connected to the hermetically sealed can. Electrodes 113, 114, and 115 are electrically connected to implantable medical device 101 via separate conductors in lead 110. Electrode 113 allows sensing of the ventricular electrogram and/or delivery of ventricular pacing pulses. Electrodes 114 and 115 allow sensing of the ventricular electrogram and/or delivery of ventricular cardioversion and defibrillation pulses.

During implantation of implantable medical device 101 into a patient, a DFT test is conducted to determine the DFT, which is the energy level required for a defibrillation pulse to terminate a VT or VF episode in the patient. To ensure successful defibrillation when needed, implantable medical device 101 is to be programmed to deliver defibrillation pulses each with an energy level that exceeds the DFT by a safety margin. During the DFT test, at least distal end of 112 of lead 110 is inserted into heart 199 such that electrodes 112, 114, and 115 are placed for sensing one or more ventricular electrograms and delivering ventricular defibrillation pulses. In one embodiment, both distal end 112 of leads 110 and distal end 107 of lead 105 are inserted into heart for the proper operation of implantable medical device 101. Implantable medical device 101 is connected to the lead(s) but not implanted during the DFT test. In one embodiment, implantable medical device 101 induces an arrhythmia episode, such as a VF episode, prior to the DFT test, and delivers one or more defibrillation pulses in attempt to terminate the induced arrhythmia episode during the DFT. In a specific embodiment, implantable medical device 101 induces a VF episode by delivering at least one defibrillation pulse during a ventricular repolarization (i.e., on a T wave).

External system 120 includes a programmer system 122, an external defibrillator system 124, and a communication link 123. Programmer system 122 communicates with implantable medical device 101 via telemetry link 103 and allows for control of the DFT test. External defibrillator system 124 provides for recovery of the patient using external defibrillation if the arrhythmia episode induced for the DFT test is not terminated by the DFT test. Communication link 123 allows for communication between programmer system 122 and external defibrillator system 124.

Telemetry link 103 is a wireless communication link providing for bidirectional data transmission between implantable medical device 101 and programmer system 122. In one embodiment, telemetry link 103 is an inductive telemetry link. In an alternative embodiment, telemetry link 103 is a far-field radio-frequency telemetry link.

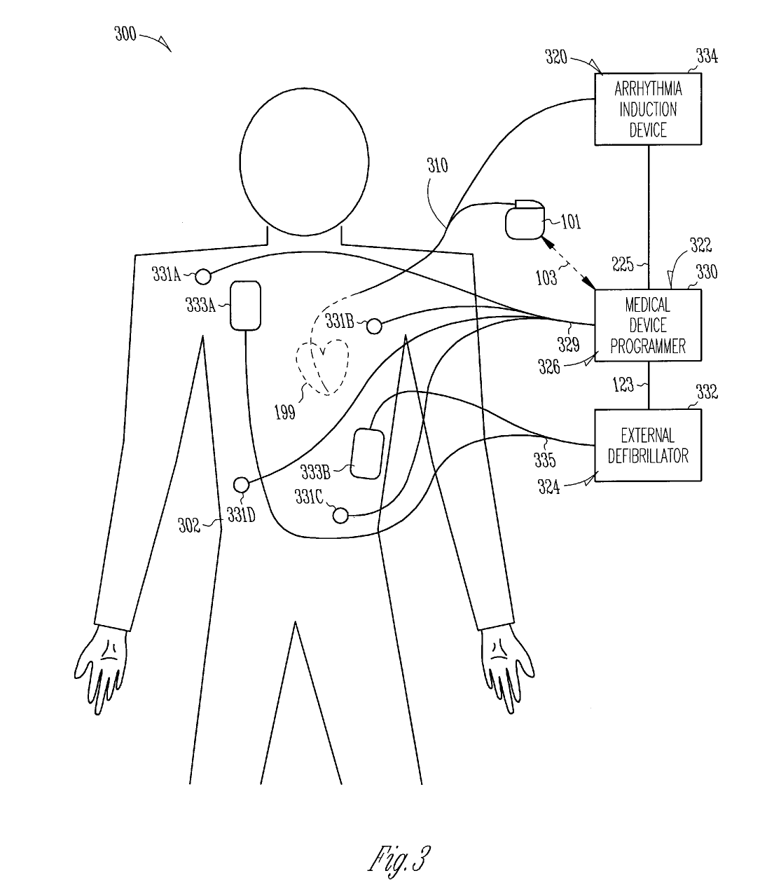

FIG. 2 is an illustration of an embodiment of a CRM system 200 and portions of the environment in which CRM system 200 operates. CRM system 200 is another embodiment of CRM system 100 and includes implantable medical device 101 that is electrically coupled to a heart 199 through one or more electrodes, such as on leads 105 and 110. An external system 220 communicates with implantable medical device 101 via a telemetry link 103.

External system 220 is another embodiment of external system 120 and includes programmer system 122, external defibrillator system 124, an arrhythmia induction system 226, communication link 123, and another communication link 225. That is, in addition to the components of CRM system 100, CRM system 200 includes arrhythmia induction system 226 and communication link 225 as part of its external system.

Arrhythmia induction system 226 allows for induction of the arrhythmia episode prior to the DFT test by delivery electrical stimulation such as cardiac pacing pulses, a low-voltage DC current, or a low-voltage AC current. Arrhythmia induction system 226 provides induction of the arrhythmia episode without consuming the battery energy of implantable medical device 101. In various embodiments, the cardiac pacing pulses, the low-voltage DC current, or the low-voltage AC current provides an alternative or supplement to the defibrillation pulse in inducing the arrhythmia episode. Cardiac pacing at a high pacing rate is a relatively painless way to induce VF in a patient, but is not uniformly successful. Delivering a DC current at about 5-12 V has a high rate of success, but may cause plating of the electrodes. Delivering an AC current of 50 or 60 Hz also has a high rate of success, but is known to cause skeletal muscular contractions. In one embodiment, arrhythmia induction system 226 is capable of delivering two or more of the cardiac pacing pulses, the low-voltage DC current, and the low-voltage AC current for inducing VF, to allow selection by the physician conducting the DFT test. Communication link 225 allows for communication between programmer system 122 and arrhythmia induction system 226.

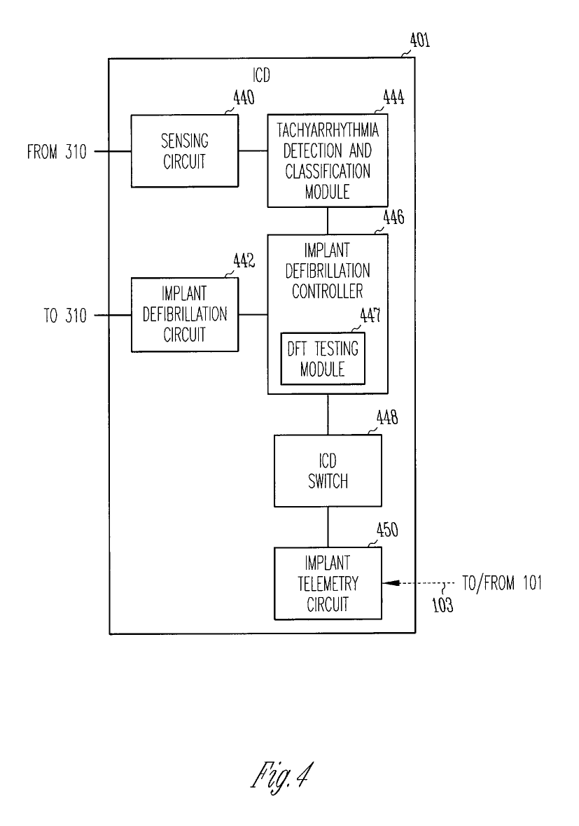

FIG. 3 is an illustration of an embodiment of a CRM system 300 and portions of the environment in which CRM system 300 operates. CRM system 300 is a specific embodiment of CRM system 200 and includes implantable medical device 101 and an external system 320. A lead system 310 electrically couples implantable medical device 101 to heart 199. In one embodiment, lead system 310 includes lead 110. In another embodiment, lead system 310 includes leads 110 and 105. External system 320 is a specific embodiment of external system 220 and includes a programmer system 322, an external defibrillator system 324, an arrhythmia induction system 326, and communication links 123 and 225. As illustrated in FIG. 3, system 300 provides for the DFT test for programming implantable medical device 101, which is to be implanted into a patient's body 302. Without arrhythmia induction system 326 and communications link 225, external system 320 as illustrated in FIG. 3 represents a special embodiment of external system 120, and CRM system 300 as illustrated in FIG. 3 represents a special embodiment of CRM system 100.

Programmer system 322 is a specific embodiment of programmer system 122 and includes a medical device programmer 330, surface ECG electrodes 331A-D, and a surface ECG cable 329 for connecting surface ECG electrodes 331A-D to medical device programmer 330. Medical device programmer 330 allows for programming of implantable medical device 101 to perform the DFT test and initiates external defibrillation if the arrhythmia episode induced prior to the DFT test sustains after the DFT test or lasts beyond a predetermined period of time (such as about 30-40 seconds). In one embodiment, medical device programmer 330 also controls the delivery of each external defibrillation pulses from external defibrillator system 324 according to an automated external defibrillation procedure. In one embodiment, surface ECG electrodes 333A-B are adhesive electrode patches for attachment to the skin of body 302 as illustrated in FIG. 3.

External defibrillator system 324 is a specific embodiment of external defibrillator system 124 and includes an external defibrillator 332, external defibrillation electrodes 333A-B, and an external defibrillation cable 335 for connecting external defibrillation electrodes 333A-B to external defibrillator 332. In one embodiment, external defibrillator 332 is an AED that performs the automated external defibrillation procedure in response to an external defibrillation command produced by medical device programmer 330. In another embodiment, external defibrillator 332 delivers each defibrillation pulses in response to a pulse delivery command produced by medical device programmer 330 according to the automated external defibrillation procedure. In one embodiment, external defibrillation electrodes 333A-B are adhesive electrode pads for attachment to the skin of body 302 as illustrated in FIG. 3.

Arrhythmia induction system 326 is a specific embodiment of arrhythmia induction system 226 and includes an arrhythmia induction device 334 coupled to heart 199 via lead system 310. Arrhythmia induction device 334 allows for induction of the arrhythmia episode prior to the DFT test. In one embodiment, medical device programmer 330 produces an arrhythmia induction command before programming implantable medical device 101 to perform the DFT test. Arrhythmia induction device 334 delivers electrical stimulation to heart 199 to induce the arrhythmia episode in response to the arrhythmia induction command. In various embodiments, arrhythmia induction device 334 delivers one or more of the cardiac pacing pulses, the low-voltage DC current, and the low-voltage AC current that are known to induce a VF episode in the patient.

Communication link 123 allows for communication between programmer system 322 and external defibrillator system 324. In one embodiment, communication link 123 is a wired link that provides for electrical connection between medical device programmer 330 and external defibrillator 332. In one embodiment, communication link 123 is a wireless link that provides for communication between medical device programmer 330 and external defibrillator 332 via telemetry.

Communication link 225 allows for communication between programmer system 322 and arrhythmia induction system 326. In one embodiment, communication link 225 is a wired link that provides for electrical connection between medical device programmer 330 and arrhythmia induction device 334. In one embodiment, communication link 225 is a wireless link that provides for communication between medical device programmer 330 and arrhythmia induction device 334.

In one embodiment, medical device programmer 330, external defibrillator 332, and arrhythmia induction device 334 are three physically separate units communicatively coupled to each other via wired and/or wireless communication links. In another embodiment, medical device programmer 330, external defibrillator 332, and arrhythmia induction device 334 are physically integrated into one external device. In another embodiment, medical device programmer 330 and external defibrillator 332 are physically integrated into one external device that is communicatively coupled to arrhythmia induction device 334 via a wired and/or wireless communication link. In another embodiment, medical device programmer 330 and arrhythmia induction device 334 are physically integrated into one external device that is communicatively coupled to external defibrillator 332 via a wired and/or wireless communication link. In another embodiment, arrhythmia induction device 334 and external defibrillator 332 are physically integrated into one external device that is communicatively coupled to medical device programmer 330 via a wired and/or wireless communication link.

In addition to controlling the DFT test, medical device programmer 330 generally allows for programming of implantable medical device 101 and receives signals acquired by implantable medical device 101. Telemetry link 103 provides for communication between medical device programmer 330 and implantable medical device 101. In one embodiment, telemetry link 103 is an inductive telemetry link. In an alternative embodiment, telemetry link 103 is a far-field radio-frequency telemetry link. Telemetry link 103 provides for data transmission from implantable medical device 101 to medical device programmer 330. This may include, for example, transmitting real-time physiological data acquired by implantable medical device 101, extracting physiological data acquired by and stored in implantable medical device 101, extracting therapy history data stored in implantable medical device 101, and extracting data indicating an operational status of implantable medical device 101 (e.g., battery status and lead impedance). Telemetry link 103 also provides for data transmission from medical device programmer 330 to implantable medical device 101. This may include, for example, programming implantable medical device 101 to acquire physiological data, programming implantable medical device 101 to perform at least one self-diagnostic test (such as for a device operational status), programming implantable medical device 101 to enable an available monitoring or therapeutic function, programming implantable medical device 101 to adjust therapeutic parameters such as pacing, cardioversion, and defibrillation parameters, programming the implantable medical device 101 to induce an arrhythmia episode for a DFT test, and programming the implantable medical device 101 to perform the DFT test.

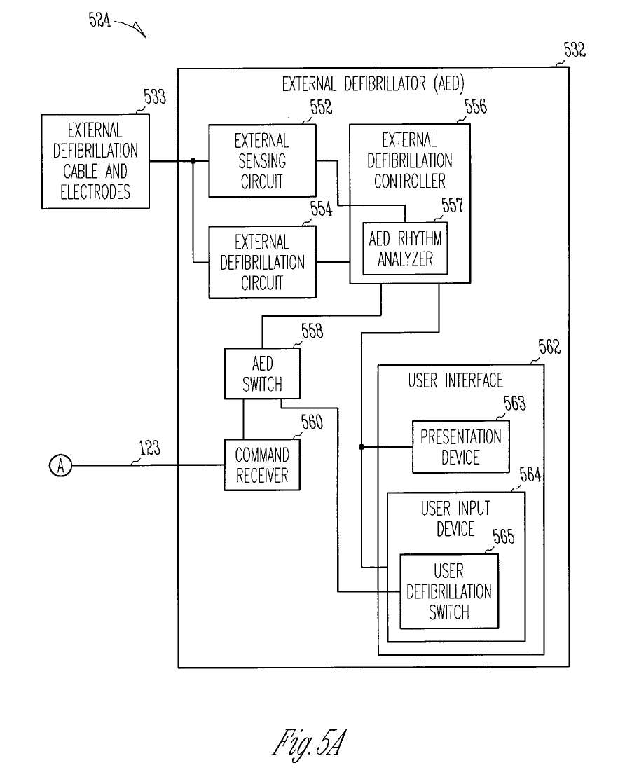

FIG. 4 is a block diagram illustrating an embodiment of an ICD 401, which is a specific embodiment of implantable medical device 101. ICD 401 includes a sensing circuit 440, an implant defibrillation circuit 442, a tachyarrhythmia detection and classification module 444, an implant defibrillation controller 446, an ICD switch 448, and an implant telemetry circuit 450.

Sensing circuit 440 senses one or more electrograms from heart 199 via lead system 310. Implant defibrillation circuit 442 delivers internal defibrillation pulses to heart 199 via lead system 310. When used in this document, “internal defibrillation pulses” refers to defibrillation pulses delivered using electrodes placed within the body of the patient, while “external defibrillation pulses” refers to defibrillation pulses delivered using electrodes attached to the skin of the patient. Tachyarrhythmia detection and classification module 444 detects tachyarrhythmia episodes and classifies each detected tachyarrhythmia episode by its type and/or origin using the one or more electrograms. In one embodiment, tachyarrhythmia detection and classification module 444 classifies the detected tachyarrhythmia episode as one of VT and SVT by comparing the morphology of the cardiac signal sensed during the detected tachyarrhythmia episode to a template morphology associated with a known cardiac rhythm such as a normal sinus rhythm (NSR). The classification of the detected tachyarrhythmia episode determines whether an internal defibrillation pulse is to be delivered based on whether the detected tachyarrhythmia episode is of a type and/or origin for which a defibrillation therapy is applicable.

Implant defibrillation controller 446 controls the delivery of the internal defibrillation pulses based on the classification of each detected tachyarrhythmia as well as in response to commands sent from medical device programmer 330. Implant defibrillation controller 446 includes a DFT testing module 447. DFT testing module 447 performs a DFT test in response to a DFT testing command. The DFT test includes delivering one or more internal defibrillation pulses each at a specified energy level. In one embodiment, the DFT test includes delivering a sequence of internal defibrillation pulses at programmed substantially different energy levels to terminate an induced arrhythmia episode. In a specific embodiment, the delivery of the sequence of internal defibrillation pulses starts with delivering an internal defibrillation pulse at the programmed minimum energy level and is stopped if the induced arrhythmia episode is terminated before the delivery of the entire sequence of internal defibrillation pulses. If a defibrillation pulse of the sequence of defibrillation pulses fails to terminate the induced arrhythmia episode, another defibrillation pulse of the sequence of defibrillation pulses at the next, stepped-up energy levels is delivered. This continues until the defibrillation pulse at the programmed maximum energy level is delivered. If the induced arrhythmia episode is not terminated after the delivery of the defibrillation pulse at the programmed maximum energy level, external defibrillation is to be performed to recover the patient.

ICD switch 448 starts and stops operation of ICD 401. In response to an ICD-off command, ICD switch stops the operation of the ICD 401. ICD 401 includes a mechanism protecting its electronic circuitry from being damaged and/or malfunctioning during external defibrillation. However, if desired, ICD switch 448 allows ICD 401 to stop its operation when external defibrillator 330 starts operating.

Implant telemetry circuit 450 transmits signals from ICD 401 to medical device programmer 330 and receives signals transmitted from medical device programmer 330 to ICD 401. Examples of the signals received using implant telemetry circuit 450 include the DFT testing command, the programmed energy levels of the internal defibrillation pulses, and the ICD-off command.

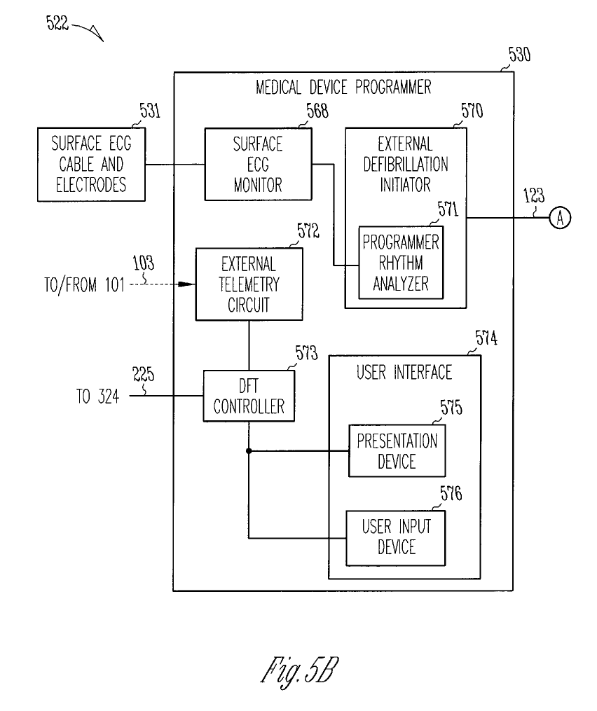

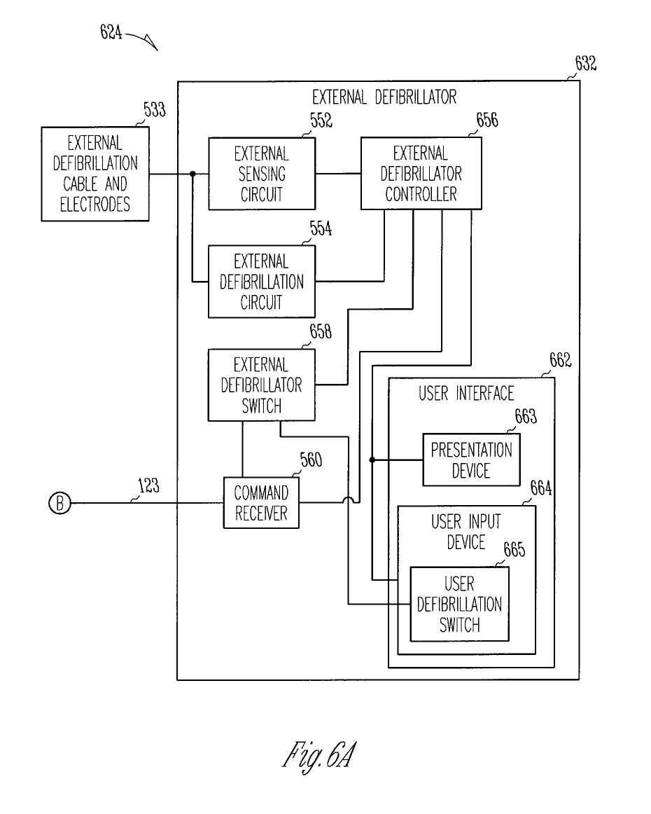

FIGS. 5A-B are each a portion of a block diagram illustrating an embodiment of an external defibrillator system 524 and a programmer system 522. In this embodiment, programmer system 522 sends the external defibrillation command to external defibrillator system 524 to start the external defibrillation. External defibrillator system 524 delivers external defibrillation pulses and controls the delivery of external defibrillation pulses according to the automated external defibrillation procedure.

FIG. 5A illustrates an embodiment of external defibrillator system 524, which is a specific embodiment of external defibrillator system 324. External defibrillator system 524 includes external defibrillation cable and electrodes 533 and an external defibrillator 532.

External defibrillation cable and electrodes 533 provide for an interface between external defibrillator 532 and body 302 through which one or more external defibrillation pulses are delivered from external defibrillator 532 to body 302. In one embodiment, external defibrillation cable and electrodes 533 include external defibrillation cable 335 and external defibrillation electrodes 333A-B as discussed above with reference to FIG. 3.

External defibrillator 532 is a specific embodiment of external defibrillator 332 and includes an external sensing circuit 552, an external defibrillation circuit 554, an external defibrillation controller 556, an AED switch 558, a command receiver 560, and a user interface 562. In the embodiment illustrated in FIG. 5A, external defibrillator 532 is an AED, which a computerized external defibrillator capable of performing the automated external defibrillation procedure. A user attaches adhesive electrode pads onto the chest wall of a patient suffering VF and/or cardiac arrest and uses a switch on the AED to start the automated external defibrillation procedure. According to the automated external defibrillation procedure, the AED senses the patient's cardiac rhythm and delivers defibrillation pulses to recover the patient while the need to defibrillate is indicated by the sensed cardiac rhythm until a predetermined number of defibrillation pulses are delivered or until a predetermined time interval has elapsed.

External sensing circuit 552 senses an external cardiac signal via external defibrillation electrodes 533. External defibrillation circuit 554 delivers the external defibrillation pulses. External defibrillation controller 556 controls the delivery of the external defibrillation pulses according to the automated external defibrillation procedure. External defibrillation controller 556 includes an AED rhythm analyzer 557 that analyzes the external cardiac signal to determine whether each external defibrillation pulse is to be delivered. In one embodiment, AED rhythm analyzer 557 detects VF. External defibrillation controller 556 causes the delivery of an external defibrillation pulse from external defibrillation circuit 554 when VF is detected. In one embodiment, external defibrillation controller 556 produces a therapy failure signal when the detected VF or cardiac arrest is still detected after the predetermined number of defibrillation pulses are delivered or after the predetermined time interval has elapsed.

AED switch 558 starts and stops operation of external defibrillator 532. In response to the external defibrillation command received from medical device programmer 330 or a user signal requesting external defibrillation, AED switch 558 starts the automated external defibrillation procedure. Command receiver 560 receives the external defibrillation command from medical device programmer 330 via communication link 123. User interface 562 includes a presentation device 563 and a user input device 564. Presentation device 563 includes a visual and/or audio indication of the states of the operation of external defibrillator 532 and a visual presentation device and/or a speaker to guide the user through the automated external defibrillation procedure. User input device 564 includes a user defibrillation switch 565, which receives the user signal requesting external defibrillation, thereby allowing the user to manually start the automated external defibrillation procedure when needed.

FIG. 5B illustrates an embodiment of a programmer system 522, which is a specific embodiment of programmer system 322. Programming system 522 includes a surface ECG cable and electrodes 531 and a medical device programmer 530.

Surface ECG cable and electrodes 531 allow for monitoring of the patient's surface ECG. In one embodiment, surface ECG cable and electrodes 531 include surface ECG cable 329 and surface ECG electrodes 331A-D, as discussed above with reference to FIG. 3.

Medical device programmer 530 is a specific embodiment of medical device programmer 330 and includes a surface ECG monitor 568, an external defibrillation initiator 570, an external telemetry circuit 572, a DFT controller 573, and a user interface 574. While components relevant to the DFT test are illustrated in FIG. 5B, medical device programmer 530 includes additional components and provides for programming of various implantable medical devices to perform various therapeutic, diagnostic, and/or monitoring functions, in various embodiments.

Surface ECG monitor 568 senses one or more surface ECG signals. In one embodiment, surface ECG monitor 568 senses a surface ECG signal using surface ECG electrodes 331A-D attached to the patient and connected to surface ECG monitor 568 via surface ECG cable 329.

External defibrillation initiator 570 produces the external defibrillation command to start the operation of external defibrillator 532. The external defibrillation command is sent to external defibrillator 532 via communication link 123. In one embodiment, when desired, external defibrillation initiator 570 also produces the ICD-off command to stop the operation of implantable medical device 101. The ICD-off command is sent to implantable medical device 101 via telemetry link 103. External defibrillation initiator 570 includes a programmer rhythm analyzer 571. Programmer rhythm analyzer 571 analyzes the surface ECG signal(s) sensed by surface ECG monitor 568 to determine whether to produce the external defibrillation command. In one embodiment, programmer rhythm analyzer 571 detects VF from a surface ECG signal when the surface ECG signal becomes available following the DFT test and produces the external defibrillation command if VF is detected.

External telemetry circuit 572 transmits signals from medical device programmer 530 to implantable medical device 101 and receives signals transmitted from implantable medical device 101 to medical device programmer 530. Implant telemetry circuit 450 and external telemetry circuit 572 form telemetry link 103.

DFT controller 573 programs implantable medical device 101 to perform the DFT test. DFT controller 573 produces the DFT testing command and transmits the DFT testing command and the programmed energy levels to implantable medical device 101 via telemetry link 103 in response to a user command for initiating the DFT test. In one embodiment, DFT controller 573 also produces the arrhythmia induction command for inducing the arrhythmia episode prior to the DFT test. The arrhythmia induction command is sent to implantable medical device 101 via telemetry link 103 and/or arrhythmia induction device 334 via communication link 225. In one embodiment, the delivery of each of the internal defibrillation pulses is controlled by DFT testing module 447 of ICD 401 (specific embodiment of implantable medical device 101), as discussed above with reference to FIG. 4. In another embodiment, the delivery of each of the internal defibrillation pulses is controlled by DFT controller 573 via telemetry link 103.

User interface 574 includes a presentation device 575 and a user input device 576. Presentation device 575 includes a display screen that displays the surface ECG signal(s) and signals received from implantable medical device 101, including the one or more electrograms. User input device 576 receives the user command for initiating the DFT test and the programmed energy levels. In one embodiment, user interface 574 includes an interactive screen that integrates portions of presentation device 575 and portions of user input device 576. In one embodiment, user interfaces 562 and 574 are each part of an integrated user interface.

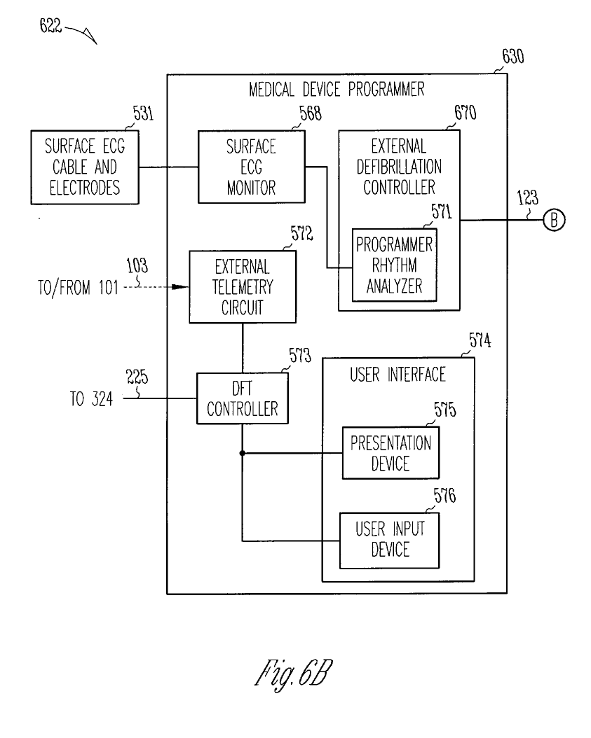

FIGS. 6A-B are each a portion of a block diagram illustrating an embodiment of an external defibrillator system 624 and a programmer system 622. In this embodiment, programmer system 622 initiates and controls the automated external defibrillation procedure by sending the pulse delivery commands to external defibrillator system 624. External defibrillator system 624 delivers the external defibrillation pulses in response to the pulse delivery commands received from programmer system 626.

FIG. 6A illustrates an embodiment of external defibrillator system 624, which is another specific embodiment of external defibrillator system 324. External defibrillator system 624 includes external defibrillation cable and electrodes 533 and an external defibrillator 632.

External defibrillator 632 is another specific embodiment of external defibrillator 332 and includes external sensing circuit 552, external defibrillation circuit 554, an external defibrillator controller 656, an external defibrillator switch 658, command receiver 560, and a user interface 662. In the embodiment illustrated in FIG. 6A, external defibrillator 632 delivers each defibrillation pulse in response to a pulse delivery command received by command receiver 560 from medical device programmer 330, or in response to a pulse delivery command received by a user defibrillation switch 665 of user interface 662.

External defibrillator controller 656 causes the delivery of each external defibrillation pulse upon receiving a pulse delivery command. External defibrillator switch 658 starts and stops operation of external defibrillator 632. In response to the external defibrillation command received from medical device programmer 330 or the user signal for turning external defibrillator 632 on, external defibrillator switch 658 activates external defibrillator 632 in preparation of receiving the pulse delivery commands and delivering the defibrillation pulses. Command receiver 560 receives the external defibrillation command from medical device programmer 330 via communication link 123. After external defibrillator 632 is turned on, command receiver 560 receives the pulse delivery command(s) from medical device programmer 330 via communication link 123.

User interface 662 includes a presentation device 663 and a user input device 664. Presentation device 663 includes a visual and/or audio indication of the states of the operation of external defibrillator 632. User input device 664 includes user defibrillation switch 665, which receives the signal for turning external defibrillator 532 on and/or the pulse delivery commands entered by the user. This allows the user to manually start and perform an external defibrillation procedure.

FIG. 6B illustrates an embodiment of a programmer system 622, which is another specific embodiment of programmer system 322. Programming system 622 includes surface ECG cable and electrodes 531 and a medical device programmer 630.

Medical device programmer 630 is a specific embodiment of medical device programmer 330 and includes surface ECG monitor 568, an external defibrillation controller 670, external telemetry circuit 572, DFT controller 573, and user interface 574. While components relevant to the DFT test are illustrated in FIG. 6B, medical device programmer 630 includes additional components and provides for programming of various implantable medical devices to perform various therapeutic, diagnostic, and/or monitoring functions in various embodiments.

External defibrillation controller 670 produces the external defibrillation command to start the performance of the automated external defibrillation procedure. The external defibrillation command is sent to external defibrillator 632 via communication link 123 to activate external defibrillator 632 and also starts the control of the delivery of one or more external defibrillation pulses from external defibrillation 632 according to the automated external defibrillation procedure using medical device programmer 630. In one embodiment, when desired, external defibrillation controller 670 also produces the ICD-off command to stop the operation of implantable medical device 101. The ICD-off command is sent to implantable medical device 101 via telemetry link 103. External defibrillation controller 670 includes programmer rhythm analyzer 571. Following the DFT test, programmer rhythm analyzer 571 analyzes the surface ECG signal(s) sensed by surface ECG monitor 568 to determine whether to produce the external defibrillation command. In one embodiment, programmer rhythm analyzer 571 detects VF from a surface ECG signal when the surface ECG signal becomes available following the DFT test and produces the external defibrillation command if VF is detected. Then, programmer rhythm analyzer 571 detects VF from the surface ECG signal following the delivery of each defibrillation pulse from external defibrillator 632 and/or before each defibrillation pulse is ready to be delivered from external defibrillator 632. If the VF is detected, external defibrillation controller 670 produces a pulse delivery command and sends the pulse delivery command to external defibrillator 632 via communication link 123.

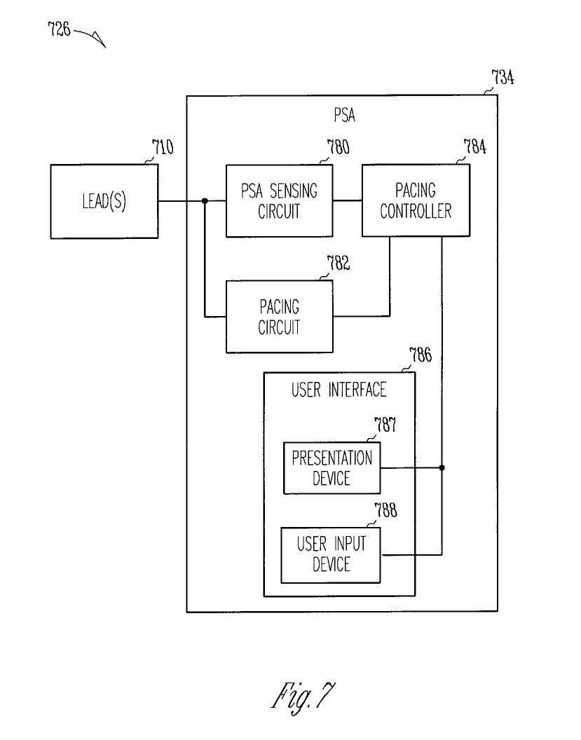

FIG. 7 is a block diagram illustrating an embodiment of an arrhythmia induction system 726, which is a specific embodiment of arrhythmia induction system 326. Arrhythmia induction system 726 includes one or more leads 710 and a pacing system analyzer (PSA) 734.

Lead(s) 710 provides the electrical connection between heart 199 and PSA 734 through which pacing pulses are delivered to heart 199. In one embodiment, lead(s) 610 include one or both of leads 110 and 105 as discussed above with reference to FIG. 1. In one embodiment, the pacing pulses are delivered from PSA 734 using the same leads through which the internal defibrillation pulses are delivered from ICD 101.

PSA 734 is a specific embodiment of arrhythmia induction device 334 and includes a PSA sensing circuit 780, a pacing circuit 782, a pacing controller 784, and a user interface 786. In one embodiment, PSA 734 is an external (non-implantable) pacing and measuring device used during implantation of an implantable pacemaker or ICD to ensure adequate lead placement, maintain basic cardiac functions, evaluate pacing parameters for an initial programming of the implantable pacemaker, and/or induce an arrhythmia episode for a DFT test. PSA sensing circuit 780 senses one or more electrograms from heart 199 via lead(s) 710. Pacing circuit 782 delivers pacing pulses to heart 199 via lead(s) 710. Pacing controller 784 controls the delivery of the pacing pulses from pacing circuit 782. User interface 786 includes a presentation device 787 and a user input device 788. Presentation device 787 displays the sensed one or more electrograms, indications of each sensed and paced event, and/or measurement parameters such as lead impedances. User input device 788 allows the user to start, stop, and adjust the delivery of the pacing pulses. For the purpose of inducing the arrhythmia episode for a DFT test, pacing controller 784 causes pacing circuit 782 to deliver the pacing pulses at a pacing rate high enough to induce fast VT or VF. In one embodiment, user interfaces 562, 574, and 786 are each part of an integrated user interface.

In various embodiments, in addition to or instead of inducing the arrhythmia episode for the DFT test, PSA 734 is used to ensure that lead(s) are properly positioned before the DFT test. It may also provide for life support when necessary during the procedure of implanting implantable medical device 101.

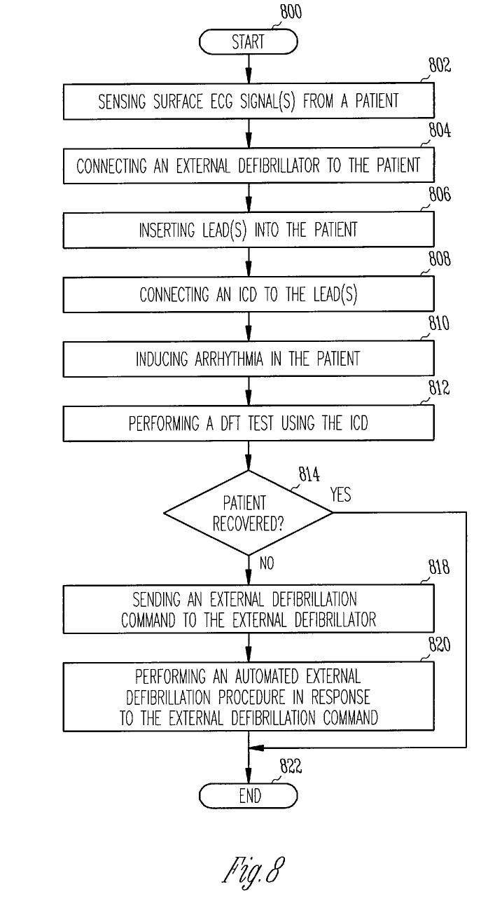

FIG. 8 is a flow chart illustrating an embodiment of a method for conducting a DFT test using an external defibrillator as a backup defibrillator. In one embodiment, the method is performed by any of CRM systems 100, 200, and 300, including various combinations of the embodiments of their components discussed above.

To perform the DFT test, a process as illustrated in FIG. 8 starts at 800, during the implantation of an ICD (or other implantable medical device with defibrillation capability) into a patient. One or more surface ECG signals are sensed from the patient at 802, using a plurality of electrodes attached onto the patient. The surface ECG signal(s) are sensed using the ECG monitoring function of a medical device programmer that communicates with the ICD via telemetry.

An external defibrillator is connected to the patient at 804. This includes attaching external defibrillation electrodes onto the patient and electrically connects the external defibrillation electrodes to the external defibrillator using a cable. The external defibrillator is activated by an external defibrillation command is produced by the medical device programmer. In one embodiment, the external defibrillator is an AED that performs an automated external defibrillation procedure in response to the external defibrillation command. In another embodiment, the external defibrillator is activated by the external defibrillation command and delivers each external defibrillation pulse in response to a pulse delivery command produced by the medical device programmer.

One or more pacing and/or defibrillation leads are inserted to the patient at 806, with electrodes for sensing, pacing, and defibrillation placed in or on the heart of the patient. The leads are then connected to the ICD, which has not been implanted, at 808.

An arrhythmia episode is induced at 810. In one embodiment, the arrhythmia episode is induced using the ICD. In a specific embodiment, a fast VT or VF episode is induced by an internal defibrillation pulse delivered from the ICD during a ventricular repolarization (i.e., on a T wave). In another embodiment, the arrhythmia episode is induced using an external arrhythmia induction device. The medical device programmer sends an arrhythmia induction command to the ICD or the external fibrillation induction to induce the arrhythmia episode.

The DFT test is performed using the ICD at 812. The medical device programmer sends a DFT testing command to the ICD to initiate the DFT test. During the DFT test, one or more internal defibrillation pulses are delivered from the ICD to terminate the induced arrhythmia episode. The one or more internal defibrillation pulses each have a programmed energy level. In one embodiment, a sequence of internal defibrillation pulses at programmed substantially different energy levels is delivered to terminate the induced arrhythmia episode. In a specific embodiment, the delivery of the sequence of internal defibrillation pulses starts with the programmed minimum energy level and stops if and when the induced arrhythmia episode is terminated before the entire sequence of internal defibrillation pulses has been delivered. If an internal defibrillation pulse of the sequence of internal defibrillation pulses fails to terminate the induced arrhythmia episode, another internal defibrillation pulse of the sequence of internal defibrillation pulses at the next, stepped-up energy levels is delivered. This continues until either the induced arrhythmic episode is terminated or the internal defibrillation pulse at the programmed maximum energy level has been delivered.

If the patient is recovered by the end of the DFT test at 814, the process illustrated in FIG. 8 ends at 822. The minimum energy level required to recover the patient from the induced arrhythmia episode (e.g., the energy level of the last internal defibrillation pulse delivered during the DFT test) is the DFT. If the patient is not recovered by the end of the DFT test at 814, the medical device programmer sends an external defibrillation command to the external defibrillator to start operation of the external defibrillator at 818. In one embodiment, if desired, the medical device programmer also sends an ICD-off command to the ICD to stop operation of the ICD. In one embodiment, following the DFT test, the one or more surface ECG signals are analyzed to determine whether the patient was recovered from the induced arrhythmia episode during the DFT test. In one embodiment, the one or more surface ECG signals are analyzed using the medical device programmer to determine whether the patient has been recovered from the induced arrhythmia episode at the end of the DFT or at the end of a predetermined period of time, whichever occurs first. The patient is not recovered if VF is detected from the one or more surface ECG signals.

In response to the external defibrillation command, an automated external defibrillation procedure is performed at 820. In one embodiment, the automated external defibrillation procedure is performed by the external defibrillator that is an AED. During the automated external defibrillation procedure, an external cardiac signal is sensed by the AED using the external defibrillation electrodes. If a need for defibrillation in indicated in the external cardiac signal, an external defibrillation pulse is delivered from the AED. After the delivery of the external defibrillation pulse, whether the induced arrhythmia episode sustains is determined. If the induced arrhythmia episode sustains, another defibrillation pulse is delivered from the AED. The AED repeats the delivery of the external defibrillation pulse and the determination of whether the induced arrhythmia episode sustains until the induced arrhythmia episode no longer sustains, until a predetermined number of defibrillation pulses have been delivered, or until a predetermined time interval has expired. In another embodiment, the automated external defibrillation procedure is performed by the external defibrillator and the medical device programmer. During the automated external defibrillation procedure, if a need for defibrillation is detected by the medical device programmer from the one or more surface ECG signals, a pulse delivery command is sent to the external defibrillator to cause the delivery of an external defibrillation pulse. After the delivery of the external defibrillation pulse, whether the induced arrhythmia episode sustains is determined by the medical device programmer. If the induced arrhythmia episode sustains, another pulse delivery command is sent to the external defibrillator to cause the delivery of another defibrillation pulse. The medical device programmer repeatedly sends the pulse delivery command to the external defibrillator and determines whether the induced arrhythmia episode sustains until the induced arrhythmia episode no longer sustains, until a predetermined number of defibrillation pulses have been delivered, or until a predetermined time interval has expired.

The process illustrated in FIG. 8 ends at 822. By automating the switching from a DFT test to external defibrillation, the present system and method reduce the risk to the patient when the DFT test fails to recover the patient from an induced arrhythmia episode. This automated switching process reduces the time required to apply external defibrillation following a DFT test, including any potential delay caused by mistakes or unfamiliarity of the physician performing the ICD implantation.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for conducting a defibrillation threshold (DFT) test for programming an implantable medical device, the system comprising:
an external defibrillator adapted to deliver one or more external defibrillation pulses in response to an external defibrillation command; and
a medical device programmer communicatively coupled to the external defibrillator, the medical device programmer adapted to program the implantable medical device to perform the DFT test and to produce the external defibrillation command if an arrhythmia episode induced prior to the DFT test sustains after the DFT test.

2. The system of claim 1, wherein at least one of the external defibrillator and the medical device programmer comprises an external defibrillation controller adapted to control the delivery of the external defibrillation pulses according to an automated external defibrillation procedure in response to the external defibrillation command.

3. The system of claim 2, wherein the external defibrillator comprises an automated external defibrillator (AED) including the external defibrillation controller.

4. The system of claim 3, wherein the external defibrillation controller comprises an AED rhythm analyzer adapted to analyze an external cardiac signal to determine whether to deliver each of the one or more external defibrillation pulses.

5. The system of claim 4, wherein the AED further comprises:
a command receiver adapted to receive the external defibrillation command;
an AED switch adapted to start the automated external defibrillation procedure when the external defibrillation command is received; and
an external defibrillation circuit adapted to deliver the one or more external defibrillation pulses.

6. The system of claim 5, furthering comprising a plurality of external defibrillation electrodes connected to the external defibrillation circuit for delivering the one or more external fibrillation pulses, and wherein the AED further comprises an external sensing circuit coupled to the plurality of external defibrillation electrodes, the external sensing circuit adapted to sense the external cardiac signal via the plurality of external defibrillation electrodes.

7. The system of claim 5, wherein the medical device programmer comprises:
a DFT controller adapted to program the ICD to perform the DFT test; and
an external defibrillation initiator adapted to produce the external defibrillation command if the arrhythmia episode induced prior to the DFT test sustains after the DFT test.

8. The system of claim 7, wherein the external defibrillation initiator comprises a programmer rhythm analyzer adapted to analyze a surface electrocardiogram (ECG) signal to determine whether to produce the external defibrillation command.

9. The system of claim 8, further comprising a plurality of surface ECG electrodes, and wherein the medical device programmer comprises a surface ECG monitor to sense the surface ECG signal using the plurality of surface ECG electrodes.

10. The system of claim 2, wherein the medical device programmer comprises the external defibrillation controller.

11. The system of claim 10, wherein the external defibrillation controller comprises a programmer rhythm analyzer adapted to analyze a surface electrocardiogram (ECG) signal to determine whether to produce the external defibrillation command.

12. The system of claim 11, wherein the external defibrillation controller is adapted to produce one or more pulse delivery commands according to the automated external defibrillation procedure, and wherein the external defibrillator comprises:
a command receiver adapted to receive the one or more pulse delivery commands;
an external defibrillation circuit adapted to deliver the one or more external defibrillation pulses; and
an external defibrillator controller adapted to cause the external defibrillation circuit to deliver one of the one or more external defibrillation pulses in response to each of the one or more pulse delivery commands.

13. The system of claim 12, wherein the programmer rhythm analyzer is further adapted to analyze the surface ECG signal to determine whether to deliver each of the pulse delivery commands.

14. The system of claim 13, further comprising a plurality of surface ECG electrodes, and wherein the medical device programmer comprises a surface ECG monitor to sense the surface ECG signal using the plurality of surface ECG electrodes.

15. The system of claim 2, wherein the medical device programmer and the external defibrillator are physically integrated into one external device.

16. The system of claim 2, further comprising an arrhythmia induction device adapted to induce the arrhythmia episode in response to an arrhythmia induction command, and wherein the medical device programmer is adapted to produce the arrhythmia induction command prior to the DFT test.

17. The system of claim 16, wherein the medical device programmer, the external defibrillator, and the arrhythmia induction device are physically integrated into one external device.

18. A method for conducting a defibrillation threshold (DFT) test for programming an implantable medical device, the method comprising:
performing the DFT test using the implantable medical device;
determining whether an arrhythmia episode induced prior to the DFT test sustains following the DFT test using a medical device programmer;
producing an external defibrillation command using the medical device programmer if the induced arrhythmia episode sustains following the DFT test;
controlling a delivery of one or more external defibrillation pulses according to an automated external defibrillation procedure using at least one of the medical device programmer and an external defibrillator in response to the external defibrillation command; and
delivering the one or more external defibrillation pulses from the external defibrillator.

19. The method of claim 18, further comprising sending an arrhythmia induction command from the medical device programmer to an external arrhythmia induction device to initiate the induction of the arrhythmia episode prior to the DFT test using the arrhythmia induction device.

20. The method of claim 18, further comprising sending a DFT testing command from a medical device programmer to the implantable medical device to initiate the DFT test, and wherein inducing the arrhythmia episode comprises inducing the arrhythmia episode by delivering an internal defibrillation pulse from the implantable medical device.

21. The method of claim 18, wherein performing the DFT test comprises delivering one or more internal defibrillation pulses each at a programmed energy level to terminate the induced arrhythmia episode.

22. The method of claim 21, wherein performing the DFT test comprises delivering a sequence of internal defibrillation pulses at substantially different programmed energy levels to terminate the induced arrhythmia episode.

23. The method of claim 22, wherein performing the DFT test comprises stopping the delivery of the sequence of internal defibrillation pulses if the induced arrhythmia is terminated before all the internal defibrillation pulses of the sequence of internal defibrillation pulses are delivered.

24. The method of claim 23, wherein performing the DFT test comprises delivering another internal defibrillation pulse of the sequence of defibrillation pulses at a stepped-up energy level following the delivery of any defibrillation pulse of the sequence of defibrillation pulses that fails to terminate the induced cardiac arrhythmia.

25. The method of claim 18, wherein determining whether the arrhythmia episode induced prior to the DFT test sustains comprises detecting ventricular fibrillation (VF) from a surface electrocardiogram (ECG) signal sensed by the medical device programmer.

26. The method of claim 18, wherein controlling the delivery of the one or more external defibrillation pulses according to the automated external defibrillation procedure comprises:
sending the external defibrillation command from the medical device programmer to the external defibrillator; and
controlling the delivery of the one or more external defibrillation pulses according to the automated external defibrillation procedure using the external defibrillator in response to the external defibrillation command.

27. The method of claim 26, wherein controlling the delivery of the one or more external defibrillation pulses according to the automated external defibrillation procedure using the external defibrillator comprises:
determining whether the induced arrhythmia episode sustains following the delivering of each of the one or more external defibrillation pulses; and
delivering another external defibrillation pulse of the one or more external defibrillation pulses if the induced arrhythmia episode sustains following the delivering of the each of the one or more external defibrillation pulses.

28. The method of claim 27, wherein determining whether the induced arrhythmia episode sustains following the delivering of the each of the one or more external defibrillation pulses comprises:
sensing an external cardiac signal using the external defibrillator via to a plurality of external defibrillation electrodes connected to the external defibrillator; and
detecting ventricular fibrillation (VF) from the external cardiac signal.

29. The method of claim 18, wherein controlling the delivery of the one or more external defibrillation pulses according to the automated external defibrillation procedure comprises:
producing one or more pulse delivery commands according to the automated external defibrillation procedure using the medical device programmer in response to the external defibrillation command; and
sending the one or more pulse delivery commands from the medical device programmer to the external defibrillator,
and wherein delivering the one or more external defibrillation pulses from the external defibrillator comprises delivering each of the one or more external defibrillation pulses in response to one of the one or more pulse delivery commands.

30. The method of claim 29, wherein producing the one or more pulse delivery commands according to the automated external defibrillation procedure using the medical device programmer comprises:
determining whether the induced arrhythmia episode sustains following the delivery of the each of the one or more external defibrillation pulses; and
producing another pulse delivery command of the one or more pulse delivery commands if the induced arrhythmia episode sustains following the delivery of the each of the one or more external defibrillation pulses.

31. The method of claim 30, wherein determining whether the induced arrhythmia episode sustains following the delivery of the each of the one or more external defibrillation pulses comprises:
sensing a surface electrocardiogram (ECG) signal using the medical device programmer connected to a plurality of surface ECG electrodes; and
detecting ventricular fibrillation (VF) from the surface ECG signal.
